(19) United States
(12) Patent Application Publication
(10) Pub. No.: US 2008/0269335 A1
(43) Pub. Date: Oct. 30, 2008

(54) Crth2 Receptor Antagonists

(76) Inventors: David Andrew Sandham, West Sussex (GB); Katharine Louise Turner, West Sussex (GB); Catherine Leblanc, West Sussex (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/568,464

(22) PCT Filed: May 3, 2005
(86) PCT No.: PCT/EP2005/004783

§ 371 Date: Oct. 30, 2006

(30) Foreign Application Priority Data
May 4, 2004 (GB) 0409921.4

(51) Int. Cl. A61K 31/19; C07C 57/30; A61P 11/06; A61P 29/00; A61P 37/08; A61P 37/06

(52) U.S. Cl. 514/571; 562/496

(57) ABSTRACT

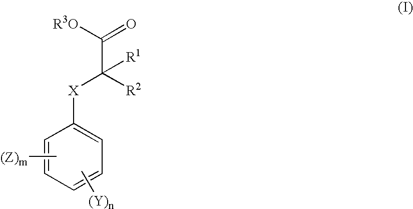

There are provided according to the invention compounds of formula (I) in free or salt form, wherein R<1>, R<2>, R<3>, X, Y, Z, m, and n are as described in the specification, process for preparing them, and their use as pharmaceuticals.

The present invention relates to organic compounds, their preparation and their use as pharmaceuticals.

In a first aspect, the present invention provides compounds of formula (I)

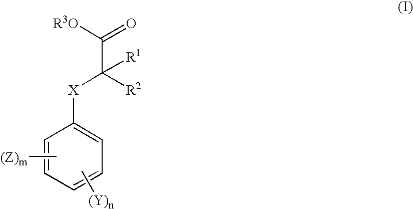

in free or salt form,
wherein
R¹ and R² are each independently H or C₁-C₈-alkyl, or together form C₃-C₈-cycloaliphatic group;
R³ is H or C₁-C₈-alkyl;
Z is

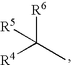

wherein
   R⁴ and R⁵ are each independently C₁-C₈-alkyl or together form a C₃-C₈-cycloaliphatic group; and
   R⁶ is H or C₁-C₈-alkyl,
or Z is a 5- to 7-membered heterocyclic ring having one or more heteroatoms selected from the group consisting of oxygen, nitrogen and sulphur,
or Z is a C₃-C₁₅-carbocyclic group;
X is O, S, SO, SO₂, CH₂ or C₁-C₈-alkylamino, e.g., C₁-C₈—NH—;
Y is halogen, cyano, nitro, carboxy, C₁-C₈-alkyl, C₁-C₈-haloalkyl, C₁-C₈-alkoxy, C₁-C₈-alkylcarbonyl, C₁-C₈-alkoxycarbonyl, C₆-C₁₀-arylcarbonyl, C₆-C₁₀-aryloxy carbonyl, C₁-C₈-alkylamino or di(C₁-C₈-alkyl)amino,
or Y is a 5- to 7-membered heterocyclic ring having one or more ring hetero atoms selected from the group consisting of oxygen, nitrogen and sulphur,
or Y is a C₃-C₁₅-carbocyclic group, optionally substituted by 1-3 groups selected from cyano, halogen, nitro, carboxy, C₁-C₈-alkyl, C₁-C₈-haloalkyl, C₁-C₈-alkoxy, C₁-C₈-alkylcarbonyl, C₁-C₈-alkoxycarbonyl, C₁-C₈-alkylamino or di(C₁-C₈-alkyl)amino;
n is an integer from 0-3; and
m is an integer from 1-2,
   for use as a pharmaceutical.

Terms used in the specification have the following meanings:

“Optionally substituted”, as used herein, means the group referred to can be substituted at one or more positions by any one or any combination of the radicals listed thereafter.

“Halogen” or “halo” may be fluorine, chlorine, bromine or iodine; preferably it is bromine or chlorine or fluorine.

“C₁-C₈-Alkyl” denotes straight-chain or branched C₁-C₈-alkyl, which may be, e.g., methyl, ethyl, n-propyl, isopropyl, n-butyl, isobutyl, sec-butyl, tert-butyl, straight- or branched-pentyl, straight- or branched-hexyl, straight- or branched-heptyl or straight- or branched-octyl. Preferably, C₁-C₈-alkyl is C₁-C₄-alkyl.

“C₃-C₁₅-Carbocyclic group”, as used herein, denotes a carbocyclic group having 3- to 15-ring carbon atoms, e.g., a monocyclic group, either cycloaliphatic, such as a C₃-C₈-cycloalkyl, e.g., cyclopropyl, cyclobutyl, cyclopentyl, cyclohexyl, cycloheptyl or cyclooctyl; or aromatic, such as phenyl; or a bicyclic group, such as bicyclooctyl, bicyclononyl including indanyl and indenyl, and bicyclodecyl including naphthyl. Preferably the C₃-C₁₅-carbocyclic group is a C₃-C₁₀-carbocyclic group, e.g., phenyl or naphthyl. The C₃-C₁₅-carbocyclic group can be substituted with 1-3 substituents or unsubstituted. Preferred substituents include halo, cyano, amino, nitro, carboxy, C₁-C₈-alkyl, halo-C₁-C₈-alkyl, C₁-C₈-alkoxy, C₁-C₈-alkylcarbonyl, C₁-C₈-alkylsulfonyl, —SO₂NH₂, (C₁-C₈-alkylamino)sulfonyl, di(C₁-C₈-alkyl)aminosulfonyl, a C₃-C₁₅-carbocyclic group and a 5- to 12-membered heterocyclic group having at least one ring heteroatom selected from nitrogen, oxygen and sulphur.

“C₃-C₈-Cycloaliphatic” denotes cycloalkyl having 3- to 8-ring carbon atoms, e.g., a monocyclic group, such as a cyclopropyl, cyclobutyl, cyclopentyl, cyclohexyl, cycloheptyl or cyclooctyl, any of which can be substituted by one or more, usually one or two, C₁-C₄-alkyl groups; or a bicyclic group, such as bicycloheptyl or bicyclooctyl. Preferably, “C₃-C₈-cycloalkyl” is C₅-C₈-cycloalkyl, i.e., cyclopentyl, cyclohexyl or cycloheptyl.

“C₁-C₈-Alkoxy” denotes straight-chain or branched C₁-C₈-alkoxy which may be, e.g., methoxy, ethoxy, n-propoxy, isopropoxy, n-butoxy, isobutoxy, sec-butoxy, tert-butoxy, straight- or branched-pentoxy, straight- or branched-hexyloxy, straight- or branched-heptyloxy or straight- or branched-octyloxy. Preferably, C₁-C₈-alkoxy is C₁-C₄-alkoxy.

“C₁-C₈-Haloalkyl” and “C₁-C₈-haloalkoxy” denotes C₁-C₈-alkyl and C₁-C₈-alkoxy as hereinbefore defined substituted by one or more halogen atoms, preferably one, two or three halogen atoms, preferably fluorine, bromine or chlorine atoms. Preferably, C₁-C₈-haloalkyl is C₁-C₄-alkyl substituted by one, two or three fluorine, bromine or chlorine atoms.

“Amino-C₁-C₈-alkyl” and “amino-C₁-C₈-alkoxy” denotes amino attached by a nitrogen atom to C₁-C₈-alkyl, e.g., NH₂—(C₁-C₈)—, or to C₁-C₈-alkoxy, e.g., NH₂—(C₁-C₈)—O—, respectively, as hereinbefore defined. Preferably, amino-C₁-C₈-alkyl and amino-C₁-C₈-alkoxy are respectively amino-C₁-C₄-alkyl and amino-C₁-C₄-alkoxy.

“Amino-(hydroxy)-C₁-C₈-alkyl” denotes amino attached by a nitrogen atom to C₁-C₈-alkyl and hydroxy attached by an oxygen atom to the same C₁-C₈-alkyl.

Preferably, amino-(hydroxy)-C₁-C₈-alkyl is amino-(hydroxy)-C₂-C₄-alkyl.

“Carboxy-C₁-C₈-alkyl” and “carboxy-C₁-C₈-alkoxy” denotes carboxy attached by a carbon atom to C₁-C₈-alkyl or C₁-C₈-alkoxy, respectively, as hereinbefore defined. Preferably, carboxy-C₁-C₈-alkyl and carboxy-C₁-C₈-alkoxy are respectively carboxy-C₁-C₄-alkyl and carboxy-C₁-C₄-alkoxy.

“C₁-C₈-Alkylcarbonyl” and “C₁-C₈-haloalkylcarbonyl” denote C₁-C₈-alkyl or C₁-C₈-haloalkyl, respectively, as hereinbefore defined, attached by a carbon atom to a carbonyl group. “C₁-C₈-alkoxycarbonyl” denotes C₁-C₈-alkoxy, as hereinbefore defined, wherein the oxygen of the alkoxy group is attached to the carbonyl carbon. Preferably, C₁-C₈-alkylcarbonyl, C₁-C₈-alkoxycarbonyl and C₁-C₈-haloalkylcarbonyl are respectively C₁-C₄-alkylcarbonyl, C₁-C₄-alkoxycarbonyl and C₁-C₄-haloalkyl-carbonyl.

“C₁-C₈-Alkylamino” and “di(C₁-C₈-alkyl)amino” denote C₁-C₈-alkyl, as hereinbefore defined, attached by a carbon atom to an amino group. The C₁-C₈-alkyl groups in di(C₁-C₈-alkyl)amino may be the same or different. Preferably, C₁-C₈-alkylamino and di(C₁-C₈-alkyl)amino are respectively C₁-C₄-alkylamino and di(C₁-C₄-alkyl)amino.

“C₁-C₈-Alkylaminocarbonyl” and “di(C₁-C₈-alkyl)aminocarbonyl” denote C₁-C₈-alkylamino and di(C₁-C₈-alkyl)amino, respectively, as hereinbefore defined, attached by a nitrogen atom to the carbon atom of a carbonyl group. Preferably, C₁-C₈-alkylaminocarbonyl and di(C₁-C₈-alkyl)-aminocarbonyl are respectively C₁-C₄-alkylaminocarbonyl and di(C₁-C₄-alkyl)-aminocarbonyl.

“Di(C₁-C₈-alkyl)amino-C₁-C₈-alkyl” and “di(C₁-C₈-alkyl)amino-C₁-C₈-alkoxy” denote di(C₁-C₈-alkyl)amino, as hereinbefore defined, attached by a nitrogen atom to the carbon atom of a C₁-C₈-alkyl or a C₁-C₈-alkoxy group, respectively. Preferably, di(C₁-C₈-alkyl)amino-C₁-C₈-alkyl and di(C₁-C₈-alkyl)amino-C₁-C₈-alkoxy are respectively di(C₁-C₄-alkyl)amino-C₁-C₄-alkyl and di(C₁-C₄-alkyl)amino-C₁-C₄-alkoxy.

“5 to 7-Membered heterocyclic ring containing at least one ring heteroatom selected from the group consisting of nitrogen, oxygen and sulphur”, as used herein, may be, e.g., furan, tetrahydrofuran, pyrrole, pyrrolidine, pyrazole, imidazole, triazole, isotriazole, tetrazole, thiadiazole, isothiazole, oxadiazole, pyridine, oxazole, isoxazole, pyrazine, pyridazine, pyrimidine, piperidine, piperazine, morpholine, triazine, oxazine or thiazole. Preferred heterocyclic rings include piperazine, morpholine, imidazole, isotriazole, pyrazole, pyridine, furan, oxazole, isoxazole and tetrazole. The 5- or 6-membered heterocyclic ring can be unsubstituted or substituted. Preferred substituents include halo, cyano, oxo, hydroxy, carboxy, nitro, C₁-C₈-alkyl, C₁-C₈-alkylcarbonyl, hydroxy-C₁-C₈-alkyl, amino-C₁-C₈-alkyl, amino(hydroxy)C₁-C₈-alkyl and C₁-C₈-alkoxy optionally substituted by aminocarbonyl. Especially preferred substituents include halo, oxo, C₁-C₄-alkyl, C₁-C₄-alkylcarbonyl, hydroxy-C₁-C₄-alkyl, amino-C₁-C₄-alkyl and amino(hydroxy)C₁-C₄-alkyl.

Throughout this specification and in the claims that follow, unless the context requires otherwise, the word “comprise”, or variations, such as “comprises” or “comprising”, will be understood to imply the inclusion of a stated integer or step or group of integers or steps but not the exclusion of any other integer or step or group of integers or steps.

In another aspect, the present invention provides compounds of formula (I) in free or salt form,

wherein
R¹ and R² are each independently H or C₁-C₈-alkyl, or together form C₃-C₈-cycloaliphatic group;
R³ is H;
Z is

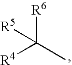

wherein
   R⁴ and R⁵ are each independently H or C₁-C₈-alkyl or together form C₃-C₈-cycloaliphatic group; and
   R⁶ is H or C₁-C₈-alkyl;
X is O, S, SO, SO₂, CH₂ or C₁-C₈-alkylamino;
Y is a C₃-C₁₅-carbocyclic group, optionally substituted by CN, NO₂, C₁-C₈-alkyl, C₁-C₈-haloalkyl, C₁-C₈-alkoxy, C₁-C₈-alkylcarbonyl, C₁-C₈-alkoxycarbonyl, C₁-C₈-alkylamino or di(C₁-C₈-alkyl)amino;
n is an integer from 0-3; and
m is an integer from 1-2,
   for use as a pharmaceutical.

In yet another aspect, the present invention provides compounds of formula (I) in free or salt form,
wherein
R¹ and R² are each independently H or C₁-C₄-alkyl;
R³ is H;
Z is

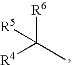

wherein
   R⁴ and R⁵ together form C₅-C₈-cycloaliphatic group; and
   R⁶ is H or C₁-C₄-alkyl;
X is O or S;
Y is a C₃-C₁₀-carbocyclic group, optionally substituted by CN, NO₂, C₁-C₄-alkyl, C₁-C₄-haloalkyl, C₁-C₄-alkoxy, C₁-C₄-alkylcarbonyl, C₁-C₄-alkoxycarbonyl, C₁-C₄-alkylamino or di(C₁-C₄-alkyl)amino;
Y is para to X and Z is ortho to X;
n is 1; and
m is 1,
   for use as a pharmaceutical.

In a yet further aspect, the present invention provides for a compound of formula (I) in free or salt form,
wherein
R¹ and R² are each independently H or C₁-C₈-alkyl, or together form C₃-C₈-cycloaliphatic group;
R³ is H or C₁-C₈-alkyl;
Z is

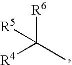

wherein
   R⁴ and R⁵ are each independently H or C₁-C₈-alkyl or together form C₃-C₈-cycloaliphatic group; and
   R⁶ is H or C₁-C₈-alkyl,
or Z is a 5- to 7-membered heterocyclic ring having one or more heteroatoms selected from the group consisting of oxygen, nitrogen and sulphur,
or Z is a C₃-C₁₅-carbocyclic group;
X is O, S, SO, SO₂, CH₂ or C₁-C₈-alkylamino;
Y is halogen, cyano, nitro, carboxy, C₁-C₈-alkyl, C₁-C₈-haloalkyl, C₁-C₈-alkoxy, C₁-C₈-alkylcarbonyl, C₁-C₈-alkoxycarbonyl, C₆-C₁₀-arycarbonyl, C₆-C₁₀-aryloxycarbonyl, C₁-C₈-alkylamino or di(C₁-C₈-alkyl)amino;
or Y is a 5- to 7-membered heterocyclic ring having one or more ring hetero atoms selected from the group consisting of oxygen, nitrogen and sulphur,
or Y is a C₃-C₁₅-carbocyclic group, optionally substituted by 1-3 groups selected from cyano, halogen, nitro, carboxy, C₁-C₈-alkyl, C₁-C₈-haloalkyl, C₁-C₈-alkoxy, C₁-C₈-alkylcarbonyl, C₁-C₈-alkoxycarbonyl, C₁-C₈-alkylamino or di(C₁-C₈-alkyl)amino;
n is an integer from 0-3; and
m is an integer from 1-2,
   with the proviso that the compound of formula (I) is not 2-cyclohexylphenoxy acetic acid, 4-chloro-2-cyclohexylphenoxy acetic acid, 4-fluoro-2-cyclohexylphenoxy acetic acid, 4-methyl-2-cyclohexylphenoxy acetic acid, 4-chloro-2-cyclopentylphenoxy acetic acid or 4-chloro-(2-(2)-allylphenoxy)acetic acid.

Preferred compounds of the present invention include compounds of formula (I) in free or salt form,

wherein
R¹ and R² are each independently H or C₁-C₈-alkyl, or together form C₃-C₈-cycloaliphatic group;
R³ is H;
Z is

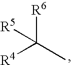

wherein
   R⁴ and R⁵ are each independently H or C₁-C₈-alkyl or together form C₃-C₈-cycloalkyl; and
   R⁶ is H or C₁-C₈-alkyl;
X is O, S, CH₂ or C₁-C₈-alkylamino;
Y is a C₃-C₁₅-carbocyclic group, optionally substituted by 1-3 groups selected from cyano, halogen, nitro, carboxy, C₁-C₈-alkyl, C₁-C₈-haloalkyl, C₁-C₈-alkoxy, C₁-C₈-alkylcarbonyl, C₁-C₈-alkoxycarbonyl, C₁-C₈-alkylamino or di(C₁-C₈-alkyl)amino;
n is an integer from 0-3; and
m is an integer from 1-2.

Yet further preferred compounds of the present invention include compounds of formula (I) in free or salt form,

wherein
R¹ and R² are each independently H or C₁-C₄-alkyl;
R³ is H;
Z is

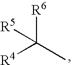

wherein
   R⁴ and R⁵ together form C₅-C₈-cycloaliphatic group; and
   R⁶ is H or C₁-C₄-alkyl;
X is O or S;
Y is a C₃-C₁₀-carbocyclic group, optionally substituted by 1-3 groups selected from cyano, halogen, nitro, carboxy, C₁-C₄-alkyl, C₁-C₄-haloalkyl, C₁-C₄-alkoxy, C₁-C₄-alkylcarbonyl, C₁-C₄-alkoxycarbonyl, C₁-C₄-alkylamino or di(C₁-C₄-alkyl)amino;
Y is para to X and Z is ortho to X;
n is 1; and
m is 1.

In a yet further aspect, the present invention provides for the use of a compound of formula (I) in any of the aforementioned embodiments, in free or salt form, for the manufacture of a medicament for the treatment of an inflammatory or allergic condition, particularly an inflammatory or obstructive airways disease.

### Salts and Isomers

Many of the compounds represented by formula (I) are capable of forming acid addition salts, particularly pharmaceutically acceptable acid addition salts. Pharmaceutically acceptable acid addition salts of the compound of formula (I) include those of inorganic acids, e.g., hydrohalic acids, such as hydrochloric acid or hydrobromic acid; nitric acid; sulfuric acid; phosphoric acid; and organic acids, e.g., aliphatic monocarboxylic acids, such as formic acid, acetic acid, caprylic acid, dichloroacetic acid, trifluoroacetic acid, hippuric acid, propionic acid and butyric acid; aliphatic hydroxy acids, such as lactic acid, citric acid, gluconic acid, mandelic acid, tartaric acid or malic acid; dicarboxylic acids, such as adipic acid, aspartic acid, fumaric acid, glutamic acid, maleic acid, malonic acid, sebacic acid or succinic acid; aromatic carboxylic acids, such as benzoic acid, p-chlorobenzoic acid, diphenylacetic acid, nicotinic acid or triphenylacetic acid; aromatic hydroxy acids, such as o-hydroxybenzoic acid, p-hydroxybenzoic acid, 1-hydroxynaphthalene-2-carboxylic acid or 3-hydroxynaphthalene-2-carboxylic acid; and sulfonic acids, such as ethanesulfonic acid, ethane-1,2-disulfonic acid, 2-hydroxyethanesulfonic acid, methanesulfonic acid, (+)-camphor-10-sulfonic acid, benzenesulfonic acid, naphthalene-2-sulfonic acid, naphthalene-1,5-disulfonic acid or p-toluenesulfonic acid. These salts may be prepared from compounds of formula (I) by known salt-forming procedures.

Compounds of formula (I) which contain acidic, e.g., carboxyl, groups, are also capable of forming salts with bases, in particular, pharmaceutically acceptable bases, such as those well-known in the art; suitable such salts include metal salts, particularly alkali metal or alkaline earth metal salts, such as sodium, potassium, magnesium calcium or zinc salts; or salts with ammonia or pharmaceutically acceptable organic amines or heterocyclic bases, such as benethamine, benzathine, diethanolamine, ethanolamine, 4(2-hydroxyethyl)morpholine, 1-(2-hydroxyethyl)pyrrolidine, N-methyl glucamine, piperazine, triethanolamine or tromethamine. These salts may be prepared from compounds of formula (I) by known salt-forming procedures.

In those compounds where there is an asymmetric carbon atom or an axis of symmetry, the compounds exist in individual optically active isomeric forms or as mixtures thereof, e.g., as racemic or diastereomeric mixtures. The present invention embraces both individual optically active R and S isomers, as well as mixtures, e.g., racemic or diastereomeric mixtures, thereof.

Specific preferred compounds of formula (I) are described hereinafter in the Examples.

The invention also provides a process for the preparation of compounds of formula (I) in free or salt form which comprises the steps of:
(a) (A) for the preparation of compounds of formula (I), where R³ is H, reacting a compound of formula (I), where R³ is C₁-C₈-alkyl

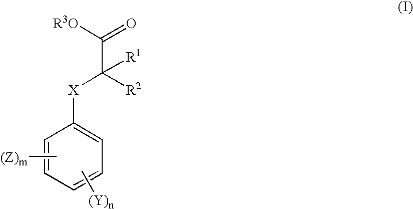

with sodium hydroxide to effect ester hydrolysis; or (B) for the preparation of compounds of formula (I), where R³ is C₁-C₈-alkyl, reacting a compound of formula (II)

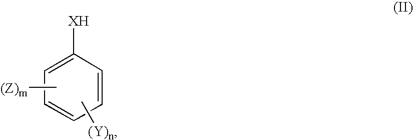

wherein R⁴, R⁵, R⁶, X (only when X is O or S), Y, Z, m and n are, as hereinbefore defined, with a compound of formula (III)

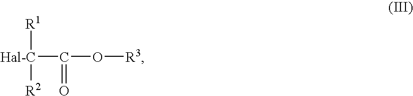

wherein
   R¹ and R² are, as hereinbefore defined; and
   R³ is C₁-C₈-alkyl; and
(b) recovering the resultant compound of formula (I) in free or salt form.

Process variant (A) may be carried out using known procedures for ester hydrolysis, or analogously, e.g., as hereinafter described in the Examples. The reaction may be carried out by reacting a compound of formula (I), wherein R³ is C₁-C₈-alkyl with aqueous sodium hydroxide in methanol at ambient temperature.

Process variant (B) may be carried out using known procedures for alkylation of phenols, or analogously, e.g., as hereinafter described in the Examples. The reaction is conveniently carried out in the presence of an inorganic base, e.g., cesium carbonate in N,N-dimethylformamide. Suitable reaction temperatures are from 10-40° C., preferably room temperature.

Compounds of formula (II), which are novel compounds, may be prepared by reacting a compound of formula (IV)

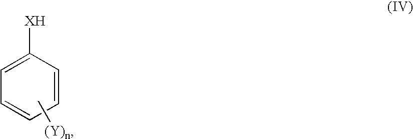

wherein X, Y and n are as hereinbefore defined, with a Friedel-Crafts alkylating reagent, e.g., using known Friedel-Crafts reaction conditions, or analogously, as hereinafter described.

For example, when Y of formula (II) is halogen and R⁶ of formula (II) is H, a compound of formula (IV) may be reacted with a cycloalkene in the presence of a Lewis Acid catalyst, e.g., cyclohexene in the presence of boron trifluoride etherate. Suitable reaction temperatures are elevated temperatures, e.g., from about 90° C. to about 120° C., but preferably about 100° C.

Alternatively, when Y of formula (II) is alkoxy and R⁶ of formula (II) is H, a compound of formula (IV) may be reacted with a cycloalkanol in the presence of a mineral acid, e.g., cylcohexanol in the presence of phosphoric acid. Suitable reaction temperatures are elevated temperatures, e.g., from about 120° C. to about 140° C., but preferably about 130° C.

Alternatively, when Y of formula (II) is halogen and R⁶ of formula (II) is alkyl, a compound of formula (IV) may be reacted with a cycloalkanol in the presence of a mineral acid and an alkylating agent, e.g., 1-methylcyclohexanol in the presence of sulphuric acid and acetic anhydride. Suitable reaction temperatures are from 10-40° C., but preferably room temperature.

### Non-Friedel-Crafts Method

For compounds where Y is CN, a compound of formula (II)

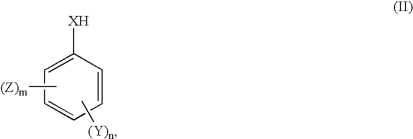

where Y is halogen, is reacted to give a compound of formula (II), where Y═CN, using known methods for the conversion of aryl halides to nitriles, e.g. reacting with copper(I) cyanide in N,N-dimethylacetamide (DMA) at 170° C.

For compounds where Y is a C₃-C₁₅-carbocyclic group, especially phenyl or substituted phenyl, a compound of formula (I), where R³ is ethyl and Y is bromine is reacted to give a compound of formula (I), where Y is 3,4-difluorophenyl, using known palladium-catalysed Suzuki cross-coupling reaction methods for the conversion of aryl halides to a biaryl system, e.g., reacting with boronic acids/esters in the presence of aqueous sodium carbonate as base and tetrakis(triphenylphosphine)palladium(0) as catalyst in tetrahydrofuran (THF) at reflux.

### Additional Non-Friedel-Crafts Method

Compounds of formula (II) may also be prepared by reacting a compound of formula (IV) where X═O with an allylic bromide in the presence of base to give the allylic ether derivative, which is then subjected to thermal Claisen rearrangement, eg using known Claisen rearrangement conditions. The resultant product is then hydrogenated to provide compounds of formula (II)

For example, when Y of formula (II) is haloalkyl, eg CF₃, a compound of formula (IV) may be reacted with an allylic bromocycloalkene in the presence of a suitable base, eg potassium carbonate in acetone. Suitable reaction temperatures are from 10-40° C., but preferably room temperature.

The product of this process is then heated, from about 120-200° C., but preferably 160° C., to undergo Claisen rearrangement.

The product of this process is treated with hydrogen in the presence of palladium catalyst. Suitable reaction temperatures are from 10-40° C., but preferably room temperature.

Compounds of formula (IV) are either commercially-available or may be obtained by known methods.

The compounds of formula (I) in free form may be converted into salt form, and vice versa, in a conventional manner. The compounds in free or salt form can be obtained in the form of hydrates or solvates containing a solvent used for crystallization. Compounds of formula (I) can be recovered from reaction mixtures and purified in a conventional manner. Isomers, such as enantiomers, may be obtained in a conventional manner, e.g., by fractional crystallization or asymmetric synthesis from correspondingly asymmetrically substituted, e.g., optically active, starting materials.

### Pharmaceutical Use and Assay

Compounds of formula (I) and their pharmaceutically acceptable salts, hereinafter referred to alternatively as agents of the invention, are useful as pharmaceuticals. In particular, the compounds have good CRTh2 receptor antagonist activity and may be tested in the following assays.

Scintillation Proximity Assay (SPA) Protocol

Membranes are prepared from K562 or Chinese hamster ovary (CHO) cells stably transfected with human CRTh2 receptors.

The assay is performed in a 96 well U-bottomed polypropylene plate in a final volume of 100 μL. For each concentration of test compound on the dose-response curve, the components of the assay are added sequentially as follows: test compound in DMSO/assay buffer (25 μL), ³H prostaglandin D₂ (25 μL) and CRTh2 membrane fragments (50 μL). The assay is incubated at ambient temperature with shaking for 60 minutes, and then harvested on to filter plates. The plate is dried for 2 hours, prior to addition of Micro-Scint 20™ (50 μL) and sealing with TopSeal-S™. Plates are then counted using a Packard Top Count instrument, each well being counted for 20 minutes. Ki values are determined using Sigma Plot™ software, using the Cheng-Prussoff equation.

CRTh2 cAMP Functional Assay Protocol

For each concentration value on the dose-response curve, test compounds are prepared in assay stimulation buffer/DMSO and 5 μL/well is added to an assay plate (384 well, white optiplate).

CHO cells stably transfected with the CRTh2 receptor are prepared (dissociated from a cell culture flask and washed in PBS) to a concentration of 4×10⁶/mL in assay stimulation buffer and added to the assay plate (10 μu/well).

The assay plate is incubated at room temperature on a shaker for 15 minutes.

A mix of agonist (10 nM Prostaglandin D₂) and 5 μM forskolin is prepared in assay stimulation buffer and added to the assay plate (5 μL/well).

In addition, a cAMP standard is serially diluted in assay stimulation buffer and added to separate empty wells on the assay plate (20 μL/well).

The assay plate is incubated at room temperature on a shaker for 60 minutes.

A cell lysis mix (lysis buffer containing Alphascreen™ donor beads and biotinylated cAMP) is prepared under darkened conditions 60 minutes prior to addition. Alphascreen™ acceptor beads are added to the lysis mix after 60 minutes. The resulting lysis mix is added to all wells of the assay plate (40 μL/well).

The assay plate is sealed with Topseal-S™ and incubated in the dark at room temperature on a shaker for 45 minutes. The plate is then counted using a Packard Fusion™ instrument.

The resulting counts per minute are converted to nM cAMP by using the prepared cAMP standard curve. IC₅₀ values are then determined using Prism™ software.

Compounds of the Examples herein below generally have Ki values in the SPA binding assay below 1 μM. For example, the compounds of Examples 2, 3, 5, 8 and 13 have Ki values of 0.060, 0.083, 0.070 0.090 and 0.021 μM, respectively.

Compounds of the Examples herein below generally have IC₅₀ values in the functional assay below 1 μM. For example, the compounds of Examples 2, 3, 5, 8 and 13 have IC₅₀ values of 0.148, 0.190, 0.138, 0.298 and 0.139 μM, respectively.

Compounds of formula (I), in free or salt form, hereinafter alternately referred to as “agents of the invention”, are antagonists of the G-protein-coupled chemoattractant receptor CRTh2, expressed on Th2 cells, eosinophils and basophils. Prostaglandin (D₂) (PGD₂) is the natural ligand for CRTh2. Thus, antagonists which inhibit the binding of CRTh2 and PGD2 are useful in the treatment of allergic and anti-inflammatory conditions. Treatment in accordance with the invention may be symptomatic or prophylactic.

Accordingly, agents of the invention are useful in the treatment of inflammatory or obstructive airways diseases, resulting, e.g., in reduction of tissue damage, airways inflammation, bronchial hyperreactivity, remodelling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitis asthma, exercise-induced asthma, occupational asthma and asthma induced following bacterial infection. Treatment of asthma is also to be understood as embracing treatment of subjects, e.g., of less than four or five years of age, exhibiting wheezing symptoms and diagnosed or diagnosable as “wheezy infants”, an established patient category of major medical concern and now often identified as incipient or early-phase asthmatics. For convenience this particular asthmatic condition is referred to as “wheezy-infant syndrome”.

Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e., therapy for or intended to restrict or abort symptomatic attack when it occurs, e.g., anti-inflammatory (e.g., corticosteroid) or bronchodilatory. Prophylactic benefit in asthma may in particular be apparent in subjects prone to “morning dipping”. “Morning dipping” is a recognized asthmatic syndrome, common to a substantial percentage of asthmatics and characterized by asthma attack, e.g., between the hours of about 4-6 a.m., i.e., at a time normally substantially distant form any previously administered symptomatic asthma therapy.

Other inflammatory or obstructive airways diseases and conditions to which the present invention is applicable include acute lung injury (ALI), adult respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular, other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis including, e.g., aluminosis, anthracosis, asbestosis, chalicosis, ptilosis, siderosis, silicosis, tabacosis and byssinosis.

Having regard to their anti-inflammatory activity, in particular in relation to inhibition of eosinophil activation, agents of the invention are also useful in the treatment of eosinophil related disorders, e.g., eosinophilia, in particular, eosinophils-related disorders of the airways, e.g., involving morbid eosinophilic infiltration of pulmonary tissues, including hypereosinophilia as it effects the airways and/or lungs, as well as, e.g., eosinophil-related disorders of the airways consequential or concomitant to Löffler's syndrome; eosinophilic pneumonia; parasitic (in particular, metazoan) infestation including tropical eosinophilia; bronchopulmonary aspergillosis; polyarteritis nodosa including Churg-Strauss syndrome; eosinophilic granuloma; and eosinophil-related disorders affecting the airways occasioned by drug-reaction.

Agents of the invention are also useful in the treatment of inflammatory or allergic conditions of the skin, e.g., psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita and other inflammatory or allergic conditions of the skin.

Agents of the invention may also be used for the treatment of other diseases or conditions, in particular, diseases or conditions having an inflammatory component, e.g., treatment of diseases and conditions of the eye, such as conjunctivitis, keratoconjunctivitis sicca and vernal conjunctivitis; diseases affecting the nose including allergic rhinitis; and inflammatory disease in which autoimmune reactions are implicated or having an autoimmune component or aetiology, including autoimmune hematological disorders, e.g., hemolytic anemia, aplastic anaemia, pure red cell anaemia and idiopathic thrombocytopenia; systemic lupus erythematosus; polychondritis; sclerodoma; Wegener granulamatosis; dermatomyositis; chronic active hepatitis; myasthenia gravis; Steven-Johnson syndrome; idiopathic sprue; autoimmune inflammatory bowel disease, e.g., ulcerative colitis and Crohn's disease; endocrine opthalmopathy; Grave's disease; sarcoidosis; alveolitis; chronic hypersensitivity pneumonitis; multiple sclerosis; primary billiary cirrhosis; uveitis (anterior and posterior); keratoconjunctivitis sicca and vernal keratoconjunctivitis; interstitial lung fibrosis; psoriatic arthritis; and glomerulonephritis with and without nephrotic syndrome, e.g., including idiopathic nephrotic syndrome or minal change nephropathy.

Other diseases or conditions which may be treated with agents of the invention include septic shock; rheumatoid arthritis; osteoarthritis; proliferative diseases, such as cancer; atherosclerosis; allograft rejection following transplantation; stroke; obesity; restenosis; diabetes, e.g., diabetes mellitus type I auvenile diabetes) and diabetes mellitus type II; diarrheal diseases; ischemia/reperfusion injuries; retinopathy, such as diabetic retinopathy or hyperbaric oxygen-induced retinopathy; and conditions characterized by elevated intraocular pressure or secretion of ocular aqueous humor, such as glaucoma.

The effectiveness of an agent of the invention in inhibiting inflammatory conditions, e.g., in inflammatory airways diseases, may be demonstrated in an animal model, e.g., a mouse or rat model, of airways inflammation or other inflammatory conditions, e.g., as described by Szarka et al., J Immunol Methods, Vol. 202, pp. 49-57 (1997); Renzi et al., Am Rev Respir Dis, Vol. 148, pp. 932-939 (1993); Tsuyuki et al., J Clin Invest, Vol. 96, pp. 2924-2931 (1995); Cernadas et al., Am J Respir Cell Mol Biol, Vol. 20, pp. 1-8 (1999); and Williams and Galli, J Exp Med, Vol. 192, pp. 455-462 (2000).

The agents of the invention are also useful as co-therapeutic agents for use in combination with other drug substances, such as anti-inflammatory, bronchodilatory or antihistamine drug substances, particularly in the treatment of obstructive or inflammatory airways diseases, such as those mentioned hereinbefore, e.g., as potentiators of therapeutic activity of such drugs or as a means of reducing required dosaging or potential side effects of such drugs. An agent of the invention may be mixed with the other drug substance in a fixed pharmaceutical composition or it may be administered separately, before, simultaneously with or after the other drug substance. Accordingly the invention includes a combination of an agent of the invention as hereinbefore described with an anti-inflammatory, bronchodilatory, anti-histamine or anti-tussive drug substance, said agent of the invention and said drug substance being in the same or different pharmaceutical composition.

Such anti-inflammatory drugs include steroids, in particular, glucocorticosteroids, such as budesonide, beclamethasone dipropionate, fluticasone propionate, ciclesonide or mometasone furoate; or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/035668, WO 03/048181, WO 03/062259, WO 03/064445 and WO 03/072592; non-steroidal glucocorticoid receptor agonists, such as those described in WO 00/00531, WO 02/10143, WO 03/082280, WO 03/082787, WO 03/104195 and WO 04/005229; LTB4 antagonists, such as those described in U.S. Pat. No. 5,451,700; LTD4 antagonists, such as montelukast and zafirlukast; PDE4 inhibitors, such as cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659 (Parke-Davis), AWD-12-281 (Asta Medica), CDC-801 (Celgene), SeICID™ CC-10004 (Celgene), KW-4490 (Kyowa Hakko Kogyo), WO 03/104204, WO 03/104205, WO 04/000814, WO 04/000839 and WO 04005258 (Merck), as well as those described in WO 98/18796 and WO 03/39544; A2a agonists, such as those described in EP 1052264, EP 1241176, EP 409595A2, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462 and WO 03/086408; A2b antagonists, such as those described in WO 02/42298; and beta (β)-2 adrenoceptor agonists, such as albuterol (salbutamol), metaproterenol, terbutaline, salmeterol, fenoterol, procaterol, and especially, formoterol and pharmaceutically acceptable salts thereof; and compounds (in free or salt or solvate form) of formula (I) of WO 00/75114, which document is incorporated herein by reference, preferably compounds of the Examples thereof, especially a compound of formula

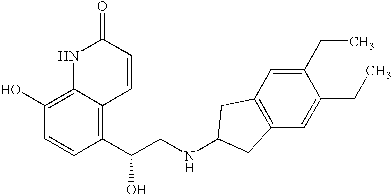

and pharmaceutically acceptable salts thereof, as well as compounds (in free or salt or solvate form) of formula (I) of WO 04/16601.

Such bronchodilatory drugs include anti-cholinergic or anti-muscarinic agents, in particular, ipratropium bromide, oxitropium bromide, tiotropium salts and CHF 4226 (Chiesi), but also those described in WO 01/04118, WO 02/51841, WO 02/53564, WO 03/00840, WO 03/87094, WO 04/05285, WO 02/00652, WO 03/53966, EP 424021, U.S. Pat. No. 5,171,744, U.S. Pat. No. 3,714,357 and WO 03/33495.

Such co-therapeutic anti-histamine drug substances include cetirizine hydrochloride, acetaminophen, clemastine fumarate, promethazine, loratidine, desloratidine, diphenhydramine and fexofenadine hydrochloride.

Combinations of agents of the invention and steroids, β-2 agonists, PDE4 inhibitors or LTD4 antagonists may be used, e.g., in the treatment of COPD or, particularly, asthma. Combinations of agents of the invention and anti-cholinergic or anti-muscarinic agents, PDE4 inhibitors, dopamine receptor agonists or LTB4 antagonists may be used, e.g., in the treatment of asthma or, particularly, COPD.

Other useful combinations of agents of the invention with anti-inflammatory drugs are those with antagonists of chemokine receptors, e.g., CCR-1, CCR-2, CCR-3, CCR-4, CCR-5, CCR-6, CCR-7, CCR-8, CCR-9, CCR-10, CXCR1, CXCR2, CXCR3, CXCR4 and CXCR5; particularly useful are CCR-3 antagonists, such as those described in WO 2002/026723, especially 4-{3-[(S)-4-(3,4-dichlorobenzyl)-morpholin-2-ylmethyl]-ureidomethyl}-benzamide and those described in WO 2003/077907 and WO 2003/007939 and WO 2002/102775.

Also especially useful are CCR-5 antagonists, such as Schering-Plough antagonists SC-351125, SCH-55700 and SCH-D; Takeda antagonists, such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770); and CCR-5 antagonists described in U.S. Pat. No. 6,166,037, WO 00/66558 and WO 00/66559.

The agents of the invention may be administered by any appropriate route, e.g., orally, e.g., in the form of a tablet or capsule; parenterally, e.g., intravenously; by inhalation, e.g., in the treatment of inflammatory or obstructive airways disease; intranasally, e.g., in the treatment of allergic rhinitis; topically to the skin, e.g., in the treatment of atopic dermatitis; or rectally, e.g., in the treatment of inflammatory bowel disease.

The present invention also provides a pharmaceutical composition comprising a compound of formula (I) in free form or in the form of a pharmaceutically acceptable salt, optionally together with a pharmaceutically acceptable diluent or carrier therefore. The composition may contain a co-therapeutic agent, such as an anti-inflammatory, bronchodilatory or anti-histamine drug as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g., patches. Compositions for inhalation may comprise aerosol or other atomizable formulations or dry powder formulations.

When the composition comprises an aerosol formulation, it preferably contains, e.g., a hydro-fluoro-alkane (HFA) propellant, such as HFA134a or HFA227 or a mixture of these, and may contain one or more co-solvents known in the art, such as ethanol (up to 20% by weight); and/or one or more surfactants, such as oleic acid or sorbitan trioleate; and/or one or more bulking agents, such as lactose. When the composition comprises a dry powder formulation, it preferably contains, e.g., the compound of formula (I) having a particle diameter up to 10 microns, optionally together with a diluent or carrier, such as lactose, of the desired particle size distribution and a compound that helps to protect against product performance deterioration due to moisture. When the composition comprises a nebulized formulation, it preferably contains, e.g., the compound of formula (I) either dissolved, or suspended, in a vehicle containing water, a co-solvent, such as ethanol or propylene glycol and a stabilizer, which may be a surfactant.

The invention includes:
(a) an agent of the invention in inhalable form, e.g., in an aerosol or other atomizable composition or in inhalable particulate, e.g., micronized form;
(b) an inhalable medicament comprising an agent of the invention in inhalable form;
(c) a pharmaceutical product comprising such an agent of the invention in inhalable form in association with an inhalation device; and
(d) an inhalation device containing an agent of the invention in inhalable form.

Dosages of agents of the invention employed in practicing the present invention will of course vary depending, e.g., on the particular condition to be treated, the effect desired and the mode of administration. In general, suitable daily dosages for oral administration are of the order of 0.01-100 mg/kg.

### EXAMPLES

Especially preferred compounds of formula (I) are also compounds of formula (V)

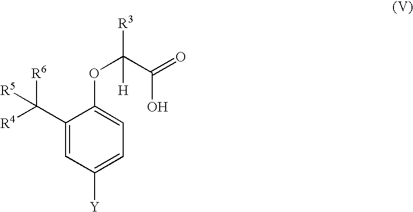

wherein R³, R⁴, R⁵, R⁶ and Y are as shown in the following Table 1, their methods of preparation being described hereinafter. Table 1 also shows characterizing mass spectrometry data.

TABLE 1

| Example | 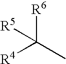 | R³ | Y | [M − H]− |
|---|---|---|---|---|
| 1 | 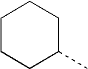 | CH₃ | Cl | 281 |
| 2 | 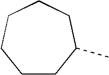 | H | Cl | 281 |
| 3 | 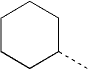 | H | Br | 312 |
| 4 | 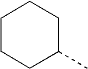 | H | OCH₃ | 263 |
| 5 | 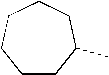 | H | Br | 326 |
| 6 | 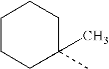 | H | Br | 326 |
| 7 | 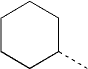 | H | CN | 258 |
| 8 | 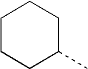 | CH₃ | Cl | 281 |
| 9 | 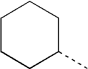 | H | 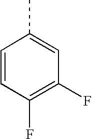 | (see NMR data) |
| 10 | 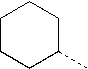 | H | 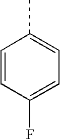 | 327 |
| 11 | 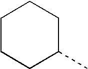 | H | 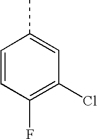 | 361/363 |
| 12 | 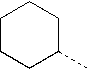 | H | 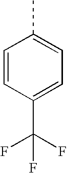 | 377 |
| 13 | 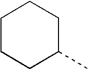 | H | CF₃ | 301 |

Note:
Example 1 is racemic mixture;
Example 8 is a single enantiomer.

### Preparation of Specific Examples

### General Experimental Conditions

LCMS are recorded on an Agilent 1100 LC system with a Waters Xterra MS C18 4.6×100 5 μM column, eluting with 5-95% 10 mM aqueous ammonium bicarbonate in acetonitrile over 10 minutes, with negative ion electrospray ionization or 5-95% water+0.1% TFA in acetonitrile with positive ion electrospray ionization. NMR are recorded at 400 MHz in CDCl₃, unless otherwise noted.

### Example 1

### (±)-2-(4-Chloro-2-cyclohexylphenoxy)propionic acid

1a) Ethyl-2-bromopropionate (1.72 g, 9.5 mmol) is added to a suspension of cesium carbonate (6.2 g, 19 mmol) and 4-chloro-2-cyclohexylphenol (2 g, 9.5 mmol) in N,N-dimethylformamide (DMF) (15 mL). The reaction is stirred for 16 hours at ambient temperature, then poured into cold 1 M aqueous HCl and extracted with ethyl acetate (EtOAc). The combined organic extracts are dried (Na₂SO₄), evaporated and purified by flash chromatography (1:8 EtOAc-isohexanes elution) to afford 2-(4-chloro-2-cyclohexylphenoxy)propionic acid ethyl ester, MH⁺ 311.

1b) 1 M aqueous NaOH (2.6 mL, 2.6 mmol) is added to a solution of -(4-chloro-2-cyclohexylphenoxy)propionic acid ethyl ester (1 g, 3.22 mmol) in methanol (4 mL). The reaction is stirred for 16 hours at ambient temperature and extracted with EtOAc. The aqueous phase is acidified to pH 5 with 2 M aqueous HCl and then extracted with EtOAc. The combined organic phases are dried (Na₂SO₄) and evaporated to afford the titled compound. [M-H]⁻ 281.

### Example 2

### (4-Chloro-2-cycloheptylphenoxy)acetic acid

2a) Cesium carbonate (1.6 g, 4.8 mmol) is added to a solution of 4-chloro-2-cycloheptylphenol [see Bangladesh J Sci Ind Res, Vol. 31, p. 1 (1996)] (0.55 g, 2.4 mmol) in DMF (2 mL), followed by ethyl bromoacetate (0.27 mL, 2.4 mmol). The reaction is stirred for 2 hours, then poured in to 1 M aqueous HCl and extracted with EtOAc. The combined organic phases are washed with water, brine, dried (Na₂SO₄) and evaporated. The crude product is purified by flash chromatography (1:10 EtOAc-isohexane elution) to afford (4-chloro-2-cycloheptyl-phenoxy)acetic acid ethyl ester.

NMR ¹H: δ 1.30 (3H t, J=7.1), 1.55-1.92 (12H, m), 3.14 (1H, m), 4.26 (2H, q, J=7.1), 4.62 (2H, s), 6.64 (1H, d, J=8.7), 7.06 (1H, dd, J=2.6-8.7), 7.18 (1H, d, J=2.6).

2b) 2 N aqueous NaOH (0.5 mL, 1 mmol) is added to a solution of (4-chloro-2-cycloheptyl-phenoxy)acetic acid ethyl ester (0.32 g, 1.03 mmol) in methanol (0.5 mL). The resultant suspension is stirred at ambient temperature for 1 hour and acidified to pH 5 with 1 M aqueous HCl. The resultant solid is filtered and dried to afford the titled compound. [M-H]⁻ 281.

### Example 3

### (4-Bromo-2-cyclohexylphenoxy)acetic acid

(4-Bromo-2-cyclohexylphenoxy)acetic acid is prepared following the same route as Example 2 by replacing 4-chloro-2-cycloheptylphenol with 4-bromo-2-cyclohexylphenol.

### Example 4

### (2-Cyclohexyl-4-methoxyphenoxy)acetic acid

4a) A mixture of 4-methoxyphenol (3 g, 24.2 mmol) and 85% phosphoric acid (2.65 g, 1.58 mL) is heated to 130° C., prior to dropwise addition of cyclohexanol (1.7 mL, 16.1 mmol) over 5 minutes. The reaction is heated for a further 1.5 hours, then cooled to ambient temperature and partitioned between water and toluene. The organic phase is dried (MgSO₄), evaporated and the crude product purified by flash chromatography (3:97 EtOAc-isohexane elution) to afford 2-cyclohexyl-4-methoxyphenol.

NMR ¹H: δ 1.38-1.50 (4H, m), 1.74-1.90 (6H, m), 2.80 (1H, m), 3.79 (3H, s), 4.48 (1H, s), 6.60 (1H, dd, J=2.5, 8.8), 6.70 (1H, d, J=8.8), 6.75 (1H, d, J=2.5).

4b) Using the general procedure of Example 2,2-cyclohexyl-4-methoxyphenol is converted to (2-cyclohexyl-4-methoxyphenoxy)acetic acid ethyl ester.

NMR ¹H: δ 1.30 (3H, d, J=7.1), 1.40-1.90 (10H, m), 3.05 (1H, m), 3.76 (3H, m), 4.26 (2H, q, J=7.1), 4.58 (2H, s), 6.62 (1H, dd, J=2.5-8.8), 6.68 (1H, d, J=8.8), 6.78 (1H, d, J=2.5).

4c) Using the general procedure of Example 2, (2-cyclohexyl-4-methoxyphenoxy)acetic acid ethyl ester is converted to the titled compound. [M-H]⁻ 263.

### Example 5

### (4-Bromo-2-cycloheptylphenoxy)acetic acid

(4-Bromo-2-cycloheptylphenoxy)acetic acid is prepared following the same route as Example 2 by replacing 4-chloro-2-heptylphenol with 4-bromo-2-cycloheptylphenol.

### Example 6

### [4-Bromo-2-(1-methylcyclohexyl)phenoxy]acetic acid

6a) Acetic anhydride (1.1 mL, 11.7 mmol) is added slowly to a mixture of 1-methylcyclohexanol (1.14 g, 10 mmol) and concentrated H₂SO₄ (0.297 mL) in heptane (5 mL), followed by 4-bromophenol (1.73 g, 10 mmol). The reaction is stirred at ambient temperature for 16 hours and the solvent is evaporated. Water is added to the residue, the pH is adjusted to 7 with saturated aqueous NaHCO₃ and the solution is extracted with ether. The organic phase is dried (MgSO₄), evaporated and purified by flash chromatography (5:1 EtOAc-isohexane elution) to afford 4-bromo-2-(1-methylcyclohexyl)phenol, [M-H]⁻ 268.

6b) Using the general procedure of Example 2,4-bromo-2-(1-methylcyclohexyl)phenol is converted to [4-bromo-2-(1-methylcyclohexyl)phenoxy]-acetic acid ethyl ester.

NMR ¹H: δ 1.28 (3H, t, J=7.1), 1.30-1.75 (8H, m), 2.1 (2H, m), 2.19 (3H, s), 4.28 (2H, q, J=7.1), 4.62 (2H, s), 6.61 (1H, d, J=8.7), 7.25 (1H, dd, J=2.5-8.7), 7.42 (1H, d, J=2.5).

6c) Using the general procedure of Example 2, [4-bromo-2-(1-methylcyclohexyl)phenoxy]-acetic acid ethyl ester is converted to the titled compound. [M-H]⁻ 327.

### Example 7

### (4-Cyano-2-cyclohexylphenoxy)acetic acid

7a) A suspension of copper (I) cyanide (0.260 g, 2.90 mmol) in N,N-DMA (0.2 mL) is heated to 170° C. and a solution of 2-cyclohexyl-4-bromophenol [see Pesticide Sci, Vol. 3, p. 575 (1972)] (0.569 g, 2.23 mol) in DMA (0.7 mL). The reaction is stirred at 170° C. for 3 hours then cooled to ambient temperature and the solvent is evaporated. The crude product is purified by flash chromatography (5:95 EtOAc-isohexane elution) to afford 3-cyclohexyl-4-hydroxybenzonitrile, [M-H]⁻ 200.

7b) Using the general procedure of Example 2,3-cyclohexyl-4-hydroxybenzonitrile is converted to (4-cyano-2-cyclohexylphenoxy)acetic acid ethyl ester, [M-H]⁻ 288.

7c) Using the general procedure of Example 2, (4-cyano-2-cyclohexylphenoxy)acetic acid ethyl ester is converted to the titled compound. [M-H]⁺ 258.

### Example 8

### (+)-2-(4-Chloro-2-cyclohexylphenoxy)propionic acid

(±)-2-(4-Chloro-2-cyclohexylphenoxy)propionic acid (Example 1) is resolved by preparative chiral HPLC using a Chiralpak AD 250 mm×4.6 mm column, eluting with 98% n-hexane and 2% isopropanol in the presence of 0.01% trifluoroacetic acid, to afford (+)-2-(4-chloro-2-cyclohexylphenoxy)propionic acid, α²⁵_{D}+3.0 (c=0.3, EtOH).

### Example 9

### (3-Cyclohexyl-3′,4′-difluoro-biphenyl-4-yloxy)-acetic acid

(4-Bromo-2-cyclohexylphenoxy)propionic acid ethyl ester is prepared following the same route as Example 3.

9a) (4-Bromo-2-cyclohexylphenoxy)propionic acid ethyl ester (0.25 g, 0.73 mmol), 3,4-difluorophenyl boronic acid (0.14 g, 0.88 mmol), tetrakis(triphenylphosphine)palladium(0) (0.042 g, 0.04 mmol) are dissolved in THF (14 mL) under argon. Sodium carbonate (Na₂CO₃) (0.22 g, 2.05 mmol) in water (1 mL) is added to the reaction mixture which is stirred at reflux overnight. The reaction mixture is filtered and the solvent is removed under reduced pressure. The residue is purified by flash chromatography (5:95 EtOAc-isohexanes elution) to afford (3-cyclohexyl-3′,4′-difluoro-biphenyl-4-yloxy)-acetic acid ethyl ester.

NMR ¹H: δ 1.24 (3H, t, J=7.1), 1.30-1.45 (4H, m), 1.65-1.90 (5H, m), 2.96-3.05 (1H, m) 4.21 (2H, q, J=7.1), 4.61 (2H, s), 6.69 (1H, d, J=8.5), 7.07-7.30 (5H, m).

9b) 4 M aqueous NaOH (1 mL, 4 mmol) is added to a solution of (3-cyclohexyl-3′,4′-difluoro-biphenyl-4-yloxy)-acetic acid ethyl ester (0.057 g, 0.15 mmol) in dioxane-water (1:1, 6 mL). The reaction is stirred for 2.5 hours at ambient temperature. The reaction mixture is acidified to pH 1 with 1 M aqueous HCl. The resultant precipitate is filtered, washed with water and dried to afford the title compound.

NMR ¹H (DMSO d₆): δ 1.20-1.58 (4H, m), 1.67-1.86 (5H, m), 2.95-3.06 (1H, m), 4.75 (2H, s), 6.88 (1H, d, J=8.5), 7.4-7.52 (4H, m), 7.69-7.78 (1H, m).

### Examples 10-12

These examples, namely (3-Cyclohexyl-4′-fluoro-biphenyl-4-yloxy)-acetic acid, (3′-Chloro-3-cyclohexyl-4′-fluoro-biphenyl-4-yloxy)-acetic acid and (3-Cyclohexyl-4′-trifluoromethyl-biphenyl-4-yloxy)-acetic acid are made by the same process as that described for Example 9, using the appropriate boronic acid

### Example 13

### (2-Cyclohexyl-4-trifluoromethyl-phenoxy)-acetic acid

Steps 13a) and 13b) are described in J. Org. Chem. 2003, 68, 9643-9647.

13a) To a solution of 4-trifluoromethyl-phenol (1.62 g, 10 mmol) in acetone (20 mL) is added potassium carbonate (1.38 g, 10 mmol) followed by 3-bromo-cyclohexene (1.61 g, 10 mmol). After refluxing the reaction mixture for 3 hours, the solid is filtered and the solvent is evaporated. The residue is purified by flash chromatography (isohexanes elution) to afford 1-(cyclohex-2-enyloxy)-4-trifluoromethyl-benzene.

NMR ¹H (CDCl₃): δ 7.56 (d, 2H, J=8.7 Hz), 7.01 (d, 2H, J=8.7 Hz), 6.07-6.01 (m, 1H), 5.91-5.85 (m, 1H), 4.93-4.87 (m, 1H), 2.29-1.60 (m, 6H)

13b) 1-(cyclohex-2-enyloxy)-4-trifluoromethyl-benzene is stirred at 150° C. for 36 hours and at room temperature for 18 hours to give 2-cyclohex-2-enyl-4-trifluoromethyl-phenol which is used as crude in the next step.

13c) To 2-cyclohex-2-enyl-4-trifluoromethyl-phenol (0.245 g, 1 mmol) in DMF (5 mL) is added cesium carbonate (0.66 g, 2 mmol) followed by ethyl bromoacetate (0.11 mL, 1 mmol). The reaction mixture is stirred overnight at room temperature, then poured in to 1 M aqueous HCl and extracted with EtOAc. The combined organic phases are washed with water, brine, dried (Na₂SO₄) and evaporated. The crude product is purified by flash chromatography (gradient from iso-hexane to 96:4 isohexane:EtOAc) to afford (2-cyclohex-2-enyl-4-trifluoromethyl-phenoxy)-acetic acid ethyl ester.

NMR ¹H (CDCl₃): δ 7.48 (d, 1H, J=2.3 Hz), 7.44 (dd, 1H, J=2.3, 8.5 Hz), 6.88 (s, 2H), 6.78 (d, 1H, J=8.5 Hz), 6.04-5.94 (m, 1H), 5.72-5.64 (m, 1H), 4.29 (q, 2H, J=7 Hz), 4.01-3.94 (m, 1H), 2.20-2.02 (m, 3H), 1.73-1.51 (m, 3H), 1.32 (t, 3H, J=7 Hz).

13d) 4 M aqueous NaOH (0.2 mL, 0.8 mmol) is added to a solution of (2-cyclohex-2-enyl-4-trifluoromethyl-phenoxy)-acetic acid ethyl ester (0.08 g, 0.24 mmol) in dioxane-water (1:1, 4 mL). The reaction is stirred for 1 hour at ambient temperature. The reaction mixture is acidified to pH 1 with 1 M aqueous HCl. The resultant precipitate is filtered, washed with water and dried to afford (2-cyclohex-2-enyl-4-trifluoromethyl-phenoxy)-acetic acid. [M-H]⁻ 299

13e) A suspension of (2-cyclohex-2-enyl-4-trifluoromethyl-phenoxy)-acetic acid (0.02 g, 0.067 mmol) and 10% Pd/C (0.035 mg) is hydrogenated for 4.5 hours. The reaction mixture is filtered on Celite™ and the solvent is evaporated to afford the title compound. [M-H]⁻ 301.

1: A compound of formula (I) 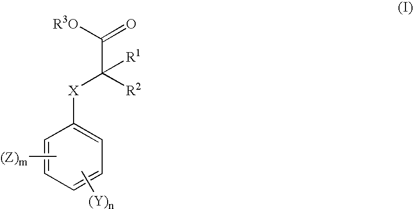
in free or salt form,
wherein
R¹ and R² are each independently H or C₁-C₈-alkyl, or together form C₃-C₈-cycloaliphatic group;
R³ is H or C₁-C₈-alkyl;
Z is 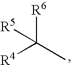
wherein
R⁴ and R⁵ are each independently C₁-C₈-alkyl or together form a C₃-C₈-cycloaliphatic group; and
R⁶ is H or C₁-C₈-alkyl,
or Z is a 5- to 7-membered heterocyclic ring having one or more heteroatoms selected from the group consisting of oxygen, nitrogen and sulphur,
or Z is a C₃-C₁₅-carbocyclic group;
X is O, S, SO, SO₂, CH₂ or C₁-C₈-alkylamino;
Y is halogen, cyano, nitro, carboxy, C₁-C₈-alkyl, C₁-C₈-haloalkyl, C₁-C₈-alkoxy, C₁-C₈-alkylcarbonyl, C₁-C₈-alkoxycarbonyl, C₆-C₁₀-arylcarbonyl, C₆-C₁₀-aryloxy carbonyl, C₁-C₈-alkylamino or di(C₁-C₈-alkyl)amino,
or Y is a 5- to 7-membered heterocyclic ring having one or more ring hetero atoms selected from the group consisting of oxygen, nitrogen and sulphur,
or Y is a C₃-C₁₅-carbocyclic group, optionally substituted by 1-3 groups selected from cyano, halogen, nitro, carboxy, C₁-C₈-alkyl, C₁-C₈-haloalkyl, C₁-C₈-alkoxy, C₁-C₈-alkylcarbonyl, C₁-C₈-alkoxycarbonyl, C₁-C₈-alkylamino or di(C₁-C₈-alkyl)amino;
n is an integer from 0-3; and
m is an integer from 1-2,
for use as a pharmaceutical.

2: A compound according to claim 1,
wherein
R¹ and R² are each independently H or C₁-C₈-alkyl, or together form C₃-C₈-cycloaliphatic group;
R³ is H;
Z is 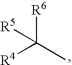
wherein
R⁴ and R⁵ are each independently H or C₁-C₈-alkyl or together form C₃-C₈-cycloaliphatic group; and
R⁶ is H or C₁-C₈-alkyl;
X is O, S, SO, SO₂, CH₂ or C₁-C₈-alkylamino;
Y is a C₃-C₁₅-carbocyclic group, optionally substituted by CN, NO₂, C₁-C₈-alkyl, C₁-C₈-haloalkyl, C₁-C₈-alkoxy, C₁-C₈-alkylcarbonyl, C₁-C₈-alkoxycarbonyl, C₁-C₈-alkylamino or di(C₁-C₈-alkyl)amino;
n is an integer from 0-3; and
m is an integer from 1-2,
for use as a pharmaceutical.

3: A compound according to claim 2,
wherein
R¹ and R² are each independently H or C₁-C₄-alkyl;
R³ is H;
Z is 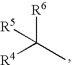
wherein
R⁴ and R⁵ together form C₅-C₈-cycloaliphatic group; and
R⁶ is H or C₁-C₄-alkyl;
X is O or S;
Y is a C₃-C₁₀-carbocyclic group, optionally substituted by CN, NO₂, C₁-C₄-alkyl, C₁-C₄-haloalkyl, C₁-C₄-alkoxy, C₁-C₄-alkylcarbonyl, C₁-C₄-alkoxycarbonyl, C₁-C₄-alkylamino or di(C₁-C₄-alkyl)amino;
Y is para to X and Z is ortho to X;
n is 1; and
m is 1,
for use as a pharmaceutical.

4: A compound of formula (I) in free or salt form,
wherein
R¹ and R² are each independently H or C₁-C₈-alkyl, or together form C₃-C₈-cycloaliphatic group;
R³ is H or C₁-C₈-alkyl;
Z is 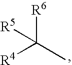
wherein
R⁴ and R⁵ are each independently H or C₁-C₈-alkyl or together form C₃-C₈-cycloaliphatic group; and
R⁶ is H or C₁-C₈-alkyl,
or Z is a 5- to 7-membered heterocyclic ring having one or more heteroatoms selected from the group consisting of oxygen, nitrogen and sulphur,
or Z is a C₃-C₁₅-carbocyclic group;
X is O, S, SO, SO₂, CH₂ or C₁-C₈-alkylamino;
Y is halogen, cyano, nitro, carboxy, C₁-C₈-alkyl, C₁-C₈-haloalkyl, C₁-C₈-alkoxy, C₁-C₈-alkylcarbonyl, C₁-C₈-alkoxycarbonyl, C₆-C₁₀-arycarbonyl, C₆-C₁₀-aryloxycarbonyl, C₁-C₈-alkylamino or di(C₁-C₈-alkyl)amino;
or Y is a 5- to 7-membered heterocyclic ring having one or more ring hetero atoms selected from the group consisting of oxygen, nitrogen and sulphur,
or Y is a C₃-C₁₅-carbocyclic group, optionally substituted by 1-3 groups selected from cyano, halogen, nitro, carboxy, C₁-C₈-alkyl, C₁-C₈-haloalkyl, C₁-C₈-alkoxy, C₁-C₈-alkylcarbonyl, C₁-C₈-alkoxycarbonyl, C₁-C₈-alkylamino or di(C₁-C₈-alkyl)amino;
n is an integer from 0-3; and
m is an integer from 1-2,
with the proviso that the compound of formula (I) is not 2-cyclohexylphenoxy acetic acid, 4-chloro-2-cyclohexylphenoxy acetic acid, 4-fluoro-2-cyclohexylphenoxy acetic acid, 4-methyl-2-cyclohexylphenoxy acetic acid, 4-chloro-2-cyclopentylphenoxy acetic acid or 4-chloro-(2-(2)-allylphenoxy)acetic acid.

5: A compound of formula (I) according to claim 4,
wherein
R¹ and R² are each independently H or C₁-C₈-alkyl, or together form C₃-C₈-cycloaliphatic group;
R³ is H;
Z is 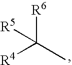
wherein
R⁴ and R⁵ are each independently H or C₁-C₈-alkyl or together form C₃-C₈-cycloalkyl; and
R⁶ is H or C₁-C₈-alkyl;
X is O, S, CH₂ or C₁-C₈-alkylamino;
Y is a C₃-C₁₅-carbocyclic group, optionally substituted by 1-3 groups selected from cyano, halogen, nitro, carboxy, C₁-C₈-alkyl, C₁-C₈-haloalkyl, C₁-C₈-alkoxy, C₁-C₈-alkylcarbonyl, C₁-C₈-alkoxycarbonyl, C₁-C₈-alkylamino or di(C₁-C₈-alkyl)amino;
n is an integer from 0-3; and
m is an integer from 1-2.

6: A compound of formula (I) according to claim 5
wherein
R¹ and R² are each independently H or C₁-C₄-alkyl;
R³ is H;
Z is 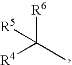
wherein
R⁴ and R⁵ together form C₅-C₈-cycloaliphatic group; and
R⁶ is H or C₁-C₄-alkyl;
X is O or S;
Y is a C₃-C₁₀-carbocyclic group, optionally substituted by 1-3 groups selected from cyano, halogen, nitro, carboxy, C₁-C₄-alkyl, C₁-C₄-haloalkyl, C₁-C₄-alkoxy, C₁-C₄-alkylcarbonyl, C₁-C₄-alkoxycarbonyl, C₁-C₄-alkylamino or di(C₁-C₄-alkyl)amino;
Y is para to X and Z is ortho to X;
n is 1; and
m is 1.

7: A compound according to claim 1 substantially as herein described with reference to any one of the Examples.

8: The use of a compound of formula (I) according to claim 1 for the manufacture of a medicament for the treatment of an inflammatory or allergic condition, particularly an inflammatory or obstructive airways disease.

9: A pharmaceutical composition comprising as active ingredient a compound according to claim 1, optionally together with a pharmaceutically acceptable diluent or carrier therefore.

10: A compound according to claim 4 for use as a pharmaceutical.

11: A process for the preparation of compounds of formula I as defined in claim 1, in free or salt form, which comprises the steps of:
(a) (A) for the preparation of compounds of formula (I) where R³ is H, reacting a compound of formula (I), where R³ is C₁-C₈-alkyl 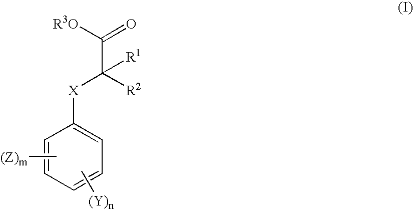
with sodium hydroxide to effect ester hydrolysis; or (B) for the preparation of compounds of formula (I) where R³ is C₁-C₈-alkyl, reacting a compound of formula (III) 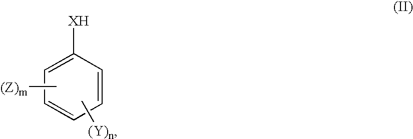
wherein R⁴, R⁵, R⁶, X (only when X is O or S), Y, Z, m and n are as hereinbefore defined, with a compound of formula (III) 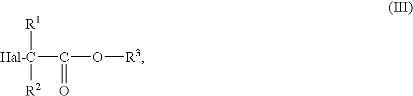
wherein
R¹ and R² are as hereinbefore defined; and
R³ is C₁-C₈-alkyl; and
(b) recovering the resultant compound of formula (I) in free or salt form.
